(19) United States
(12) Patent Application Publication
(10) Pub. No.: US 2019/0288565 A1
(43) Pub. Date: Sep. 19, 2019

(54) RF POWER TRANSFER COIL FOR IMPLANTABLE VAD PUMPS

(71) Applicant: Medtronic, Inc., Minneapolis, MN (US)

(72) Inventors: Gonzalo MARTINEZ, Mendota Heights, MN (US); David J. PEICHEL, Minneapolis, MN (US)

(21) Appl. No.: 16/295,412

(22) Filed: Mar. 7, 2019

Related U.S. Application Data
(60) Provisional application No. 62/642,766, filed on Mar. 14, 2018.

(51) Int. Cl. H02J 50/20; H02J 50/10; H01F 38/14; A61M 1/12; A61M 1/10

(52) U.S. Cl. CPC H02J 50/20; H02J 50/10; H01F 38/14; H02J 7/025; A61M 1/1086; A61M 1/127; A61M 1/122

(57) ABSTRACT

An implantable radiofrequency receiving coil configured to electrically couple with a radiofrequency source coil for transcutaneous energy transfer. The receiving coil includes at least one copper conductor defining a coil and configured to power an implantable blood pump. The at least one copper conductor is coated with tantalum.

### CROSS-REFERENCE TO RELATED APPLICATION

This application claims the benefit of U.S. application Ser. No. 62/642,766, filed Mar. 14, 2018 entitled RF POWER TRANSFER COIL FOR IMPLANTABLE VAD PUMPS.

### FIELD

The present technology generally relates to an implantable radiofrequency receiving coil for a transcutaneous energy transfer system (TETS).

### BACKGROUND

Transcutaneous energy transfer (TET) systems are used to supply power to devices such as heart pumps implanted internally within a human body. An electromagnetic field generated by a transmitting coil outside the body can transmit power across a cutaneous (skin) barrier to a magnetic receiving coil implanted within the body. The receiving coil can then transfer the received power to the implanted heart pump or other internal device and to one or more batteries implanted within the body.

One of the challenges with TET systems are the material properties of the receiving coil and the resultant side effects on the patient. Currently, wires that are implanted within a patient for receiving energy are composed of a silver or silver alloy material to conduct energy. Such wires, while having high conductivity, have a relatively high resistance at higher frequencies due to skin effects and are corrosive. The high resistance, particularly at radio frequencies necessary for high power levels, may increase the prevalence of patient burns and/or discomfort. The high corrosiveness means that any silver-based implanted coil would typically require a hermetic package to reduce corrosiveness, but lowers conductivity and increases cost.

### SUMMARY

The techniques of this disclosure generally relate to an implantable radiofrequency receiving coil for a transcutaneous energy transfer system (TETS).

The details of one or more aspects of the disclosure are set forth in the accompanying drawings and the description below. Other features, objects, and advantages of the techniques described in this disclosure will be apparent from the description and drawings, and from the claims.

The present invention advantageously provides for an implantable radiofrequency receiving coil configured to electrically couple with a radiofrequency source coil for transcutaneous energy transfer. The receiving coil includes at least one copper conductor defining a coil and configured to power an implantable blood pump. The at least one copper conductor is coated within tantalum.

In another aspect of this embodiment, the at least one copper conductor includes a plurality of copper conductors, each of the plurality of conductors being coated within tantalum and being insulated from an adjacent one of the plurality of conductors.

In another aspect of this embodiment, the receiving coil defines a Litz wire.

In another aspect of this embodiment, the tantalum completely surrounds the at least one copper conductor.

In another aspect of this embodiment, the at least one copper conductor is entirely composed of copper.

In another aspect of this embodiment, the tantalum includes tantalum pentoxide.

In another embodiment, a transcutaneous energy transfer system for powering an implantable medical device includes a source coil positionable on a patient's skin. A battery is electrically coupled to the source coil. The source coil is configured to transfer electrical energy through the patient's skin. A receiving coil is implantable within the patient. The receiving coil is configured to receive the energy transferred by the source coil, the receiving coil including at least one copper conductor defining a coil and configured to power the implantable medical device, the at least one copper conductor being coated within one from the group consisting of graphene and tantalum. The implantable medical device is electrically coupled to the receiving coil.

In another aspect of this embodiment, the at least one copper conductor includes a plurality of copper conductors, each of the plurality of conductors being coated with tantalum and being insulated from an adjacent one of the plurality of conductors.

In another aspect of this embodiment, the receiving coil defines a Litz wire.

In another aspect of this embodiment, each of the plurality of conductors is coated with tantalum, and wherein the tantalum completely surrounds the at least one copper conductor.

In another aspect of this embodiment, the at least one copper conductor is entirely composed of copper.

In another aspect of this embodiment, the tantalum includes tantalum pentoxide.

In another aspect of this embodiment, the implantable medical device is an implantable blood pump.

In another aspect of this embodiment, the implantable blood pump is electrically coupled to a controller implanted within the body, the controller being configured to control operation of the implantable blood pump.

In another aspect of this embodiment, the controller is electrically coupled to the receiving coil.

In another aspect of this embodiment, the controller is powered by the receiving coil.

In another aspect of this embodiment, the receiving coil is disposed in a non-hermetic package.

In another aspect of this embodiment, receiving coil does not include welds and joints.

In yet another embodiment, a transcutaneous energy transfer system for powering an implantable blood pump includes a substantially planar source coil positionable on a patient's skin. A battery is electrically coupled to the source coil. The source is being configured to transfer electrical energy through the patient's skin into a body of the patient. A receiving coil is implantable within the patient. The receiving coil is configured to receive the energy transferred by the source coil. The receiving coil includes a plurality of copper conductors defining a substantially planar coil and configured to power and electrically couple with the implantable blood pump, each the plurality of copper conductors being coated within tantalum pentoxide and defining a Litz configuration without welds and joints. A controller is implantable within the patient and electrically coupled to the battery and to the receiving coil, the controller is configured to control operation of the implantable blood pump.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front inside of the body view of a patient with a left ventricular assist device, receiving coil, and controller, fully implanted within the patient;

FIG. 2 is a front outside of the body view of the patient shown in FIG. 1 showing a battery and transmission coil coupled to the patient;

FIG. 3 is a front view of the receiving coil and controller shown in FIG. 1;

FIG. 3A is a zoomed in view of an embodiment of a first end of the receiving coil shown in FIG. 3;

FIG. 3B is a zoomed in view of another embodiment of a first end of the receiving coil shown in FIG. 3;

FIG. 4 is a cross-sectional view of a portion of the receiving coil shown in FIG. 3; and

FIG. 5 is a cross-sectional view of another embodiment of a receiving coil of the present application.

### DETAILED DESCRIPTION

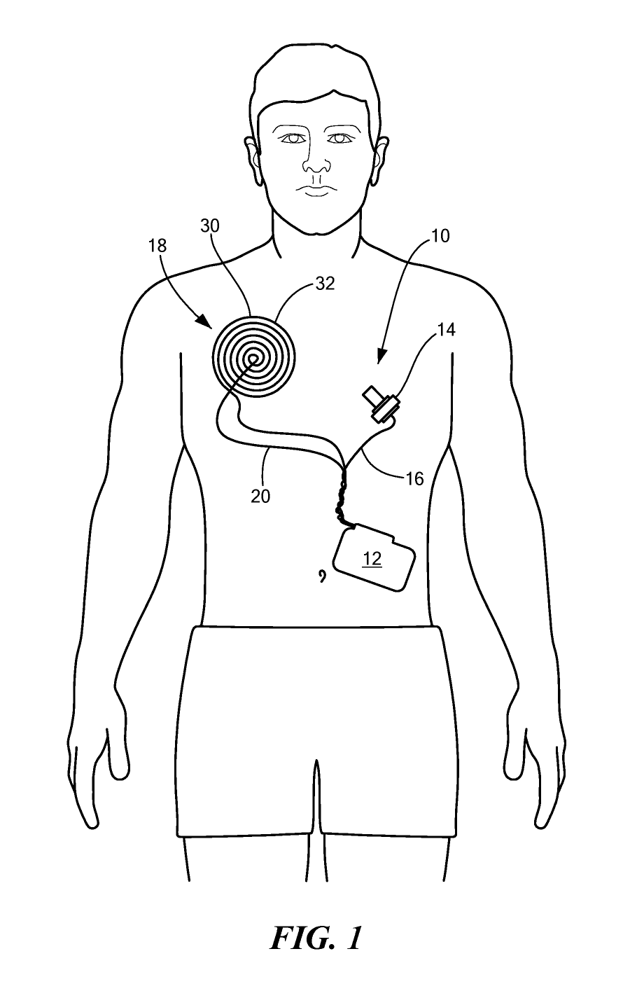

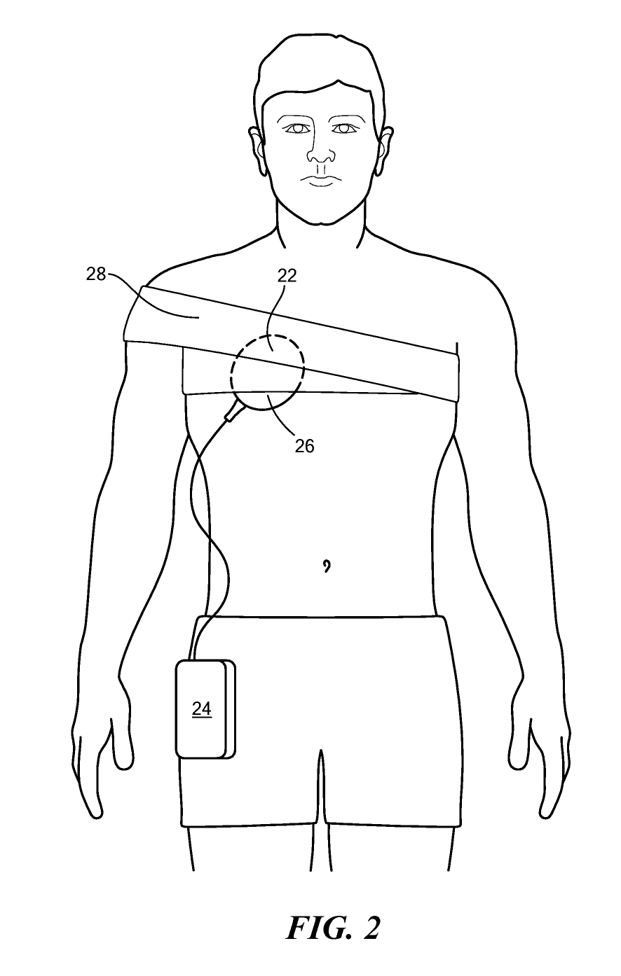

Referring now to the drawings in which like reference designators refer to like elements there is shown in FIGS. 1 and 2 an exemplary transcutaneous energy transfer (TET) system constructed in accordance with the principles of the present application and designated generally as “10.” The system 10 is fully implantable within a patient, whether human or animal, which is to say there are no percutaneous connections between implanted components of the system 10 and components outside of the body of the patient. In the configuration shown in FIG. 1, the system 10 includes a controller 12 implanted within the body of the patient. The controller 12 may include a battery (not shown) configured to power the components of the controller and provide power one or more implantable medical device, for example, a ventricular assist device (VAD) 14 implanted within the left ventricle of the patient's heart. VADs 14 may include centrifugal pumps, axial pumps, or other kinds electromagnetic pumps configured to pump blood from the heart to blood vessels to circulate around the body. One such centrifugal pump is the HVAD sold by HeartWare, Inc. and is shown and described in U.S. Pat. No. 7,997,854 the entirety of which is incorporated by reference. One such axial pump is the MVAD sold by HeartWare, Inc. and is shown and described in U.S. Pat. No. 8,419,609 the entirety of which is incorporated herein by reference. In an exemplary configuration, the VAD 14 is electrically coupled to the controller 12 by one or more implanted conductors 16 configured to provide power to the VAD 14, relay one or more measured feedback signals from the VAD 14, and/or provide operating instructions to the VAD 14.

Continuing to refer to FIG. 1, a receiving coil 18 may also be coupled to the controller 12 by, for example, one or more implanted conductors 20. In an exemplary configuration, the receiving coil 18 may be implanted subcutaneously proximate the thoracic cavity, although any subcutaneous position may be utilized for implanting the receiving coil 18. The receiving coil 18 is configured to be inductively powered through the patient's skin by a transmission coil 22 (seen in FIG. 2) disposed opposite the receiving coil 18 on the outside of the patient's body. For example, as shown in FIG. 2, a transmission coil 22 may be coupled to a power source 24, for example a portable battery carried by the patient. In one configuration, the battery is configured to generate a radiofrequency signal for transmission of energy from the transmission coil 22 to the receiving coil 18. In the configuration shown in FIG. 2, the transmission coil 22 is optionally housed within sealed packaging 26 to protect the transmission coil 22 and is optionally attached to a sling 28 around the patient's torso to maintain the transmission coil 22 in a fixed position for power transmission to the receiving coil 18. Although a sling 28 is shown in FIG. 2, any fixation device may be utilized to either adhere or otherwise affix the transmission coil 22 to the skin of the patient. The transmission coil 22 may be composed of conductive alloy, for example, copper with a sufficient number of turns and conductivity to transmit power sufficient to power the VAD 14, for example, 2-10 W. In other configuration, the conductive alloy may be gold, palladium, silver, or other metals.

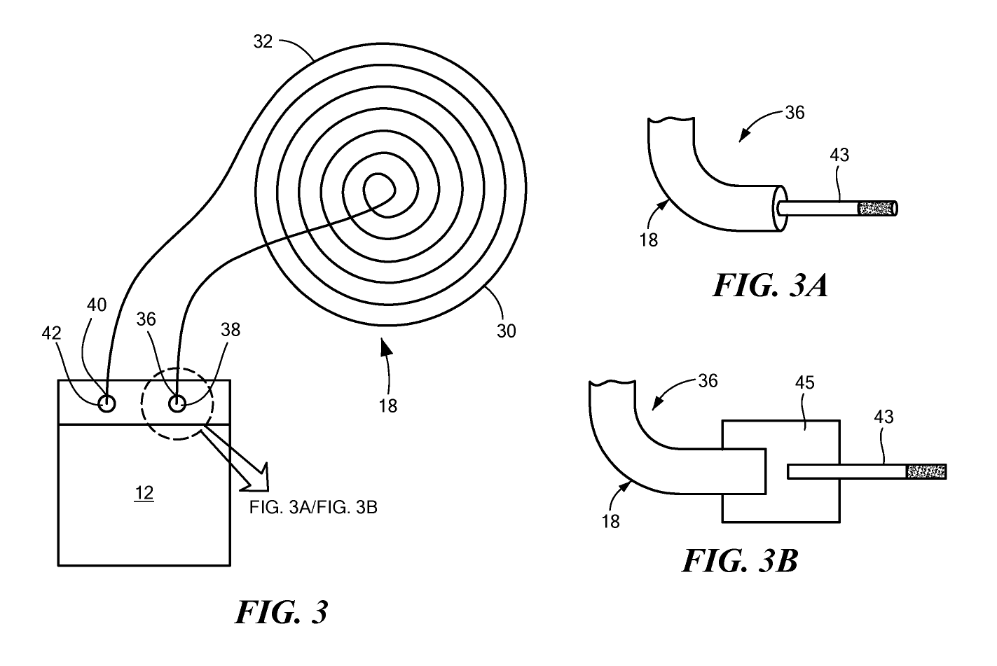

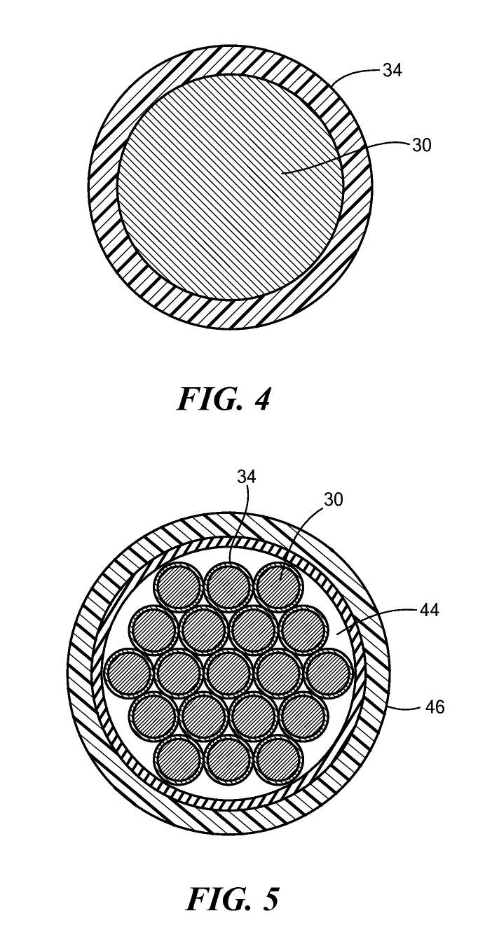

Referring now to FIGS. 1 and 3, the receiving coil 18 includes at least one copper conductor 30 defining a coil 32 and configured to power the VAD 12. In one configuration, the at least one copper conductor 30 is coated within a corrosion resistant material 34 (FIG. 4), such as tantalum or graphene. Other corrosion resistant material may include but are not limited to niobium, titanium, platinum, gold, and other high corrosion resistance metals, metal alloys, ceramics, and composites, for example, sputtered metals, graphene, and other coatings. In one configuration an electrically insulating material, for example, ETFE, may further be coated on the corrosion resistant material 34, either partially or completely surrounding both the coil 32 and the corrosion resistant material 34. In one configuration, the receiving coil 18 is defines a substantially planar coil defining a diameter of 4-10 cm such that is substantially co-planer with an interior surface of the dermis. The at least one copper conductor 30 may be solid-core, entirely composed of copper, and is corrosive resistant, thus reducing or eliminating the need for the receiving coil 18 to be packaged within a hermetically sealed material. In other configurations, the at least one copper conductor may be substantially composed of copper but may include other metal or metallic alloys, such as silver. In one configuration, the at least one copper conductor 30 is between 10-24 AGW and defines between 6-14 turns to define coil 32. In an exemplary configuration, the at least one copper conductor 30 is 14 AWG or less and defines 10 turns without any welds or joints. The at least one copper conductor 30 may also be stranded or braided. For example, the at least one copper conductor 30 may be 14 AWG and include a plurality of copper wires defining the same cross-sectional area. A first end 36 of the coil 18 may be electrically coupled to a first coupling 38 of the controller 12 and a second end 40 of the coil 18 may be coupled to a second coupling 42 of the controller 12 such that a voltage may be applied to the coil 18. In such a construction, the coil 18 does not include any joints, but rather smooth turns.

Referring now to FIG. 3A, to isolate the copper first end 36 and the second end 40 of the coil 18 from the patient's body, the first end 36 and the second end 40 the may be etched with an etching material, for example, ferric chloride, HNO₃ and a biocompatible wire or pin is inserted within the coil 18. For example, a pin 43 composed of a biocompatible conductive material, for example Niobium or Titanium, may extend distally away from the ends 36 and 40, which may be seam welded to isolate the copper of coil 18 from the patient. The pin 43 may further enclosed or otherwise coated with sapphire or ceramic for coupling with the first or second couplings 38 and 42 respectively. The distal end of the pin 43 may optionally be sputtered with gold such that the distal end of the pin 43 may be soldered to the first or second couplings 38 and 42.

Referring now to FIG. 3B, in another configuration, first end 36 and the second end 40 of the coil 18 may be crimp welded without any etching to the ends 36 and 40. For example, each end 36 and 40 may be crimp welded to and within biocompatible material crimp material 45, for example, Platinum, Iridium, Gold, Titanium, etc. The pin 43 may extend distally from the crimp material 45 for soldering or otherwise engagement to the first or second couplings 38 and 42 respectively. Referring now to FIG. 4, in an exemplary configuration, the at least one copper conductor 30 is entirely coated and disposed within the corrosion resistant material 34, which is tantalum pentoxide. The cross-sectional area of the at least one copper conductor 30 is larger than the cross-sectional area of the corrosion resistant material 34, which increases conductivity. For example, the thickness of corrosion resistant material 34 may range from 0.1 mm thick to 2 mm thick, and in some configurations, up to 5 mm thick. In an exemplary configuration, the tantalum pentoxide is extruded or otherwise deposited on the surface of the at least one copper conductor 30 and forms a substantially uniform layer around the at least one copper conductor 30. In such a configuration, the coil 18 is corrosive resistant and biocompatible without a hermetically sealed package. That is, the coil 18 may be implanted underneath the skin without any packaging around the coil 18. Owing to the creation of a thin oxide layer around the at least one copper conductor 30 from the tantalum pentoxide layer, the coil 18 may optionally define a Litz type wire 44 construction, shown in FIG. 5. For example, as shown in FIG. 5, a plurality of at least one copper conductors 30, each being clad within corrosion resistant material 34 may be disposed within a larger outer corrosion resistant material 46, which house the components of the Litz type wire 44, and each copper conductor 30 may be insulated from an adjacent copper conductor 30. In such a configuration, the skin effect is reduced, lowering the overall series resistance, thus reducing the amount of heat generated. Any Litz configuration may be utilized in forming coil 18, for example, Type 1-8 Litz wire configurations, with stranded or solid core copper conductors 30. In an exemplary configuration, the Litz wire 46 may be a 14 AWG and may be composed of any number of conductors 30.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An implantable radiofrequency receiving coil configured to electrically couple with a radiofrequency source coil for transcutaneous energy transfer, the receiving coil comprising:
at least one copper conductor defining a coil and configured to power an implantable blood pump, the at least one copper conductor being coated with tantalum.

2. The receiving coil of claim 1, wherein the at least one copper conductor includes a plurality of copper conductors, each of the plurality of conductors being coated with tantalum and being insulated from an adjacent one of the plurality of conductors.

3. The receiving coil of claim 2, wherein the receiving coil defines a Litz wire.

4. The receiving coil of claim 1, wherein the tantalum completely surrounds the at least one copper conductor.

5. The receiving coil of claim 1, wherein the at least one copper conductor is entirely composed of copper.

6. The receiving coil of claim 1, wherein the tantalum includes tantalum pentoxide.

7. A transcutaneous energy transfer system for powering an implantable medical device, comprising:
a source coil positionable on a patient's skin;
a battery electrically coupled to the source coil, the source coil being configured to transfer electrical energy through the patient's skin;
a receiving coil implantable within the patient, the receiving coil being configured to receive the energy transferred by the source coil, the receiving coil including at least one copper conductor defining a coil and configured to power the implantable medical device, the at least one copper conductor being coated with one from the group consisting of graphene and tantalum; and
the implantable medical device being electrically coupled to the receiving coil.

8. The system of claim 7, wherein the at least one copper conductor includes a plurality of copper conductors, each of the plurality of conductors being coated with tantalum and being insulated from an adjacent one of the plurality of conductors.

9. The system of claim 8, wherein the receiving coil defines a Litz wire.

10. The system of claim 7, wherein each of the plurality of conductors is coated with tantalum, and wherein the tantalum completely surrounds the at least one copper conductor.

11. The system of claim 7, wherein the at least one copper conductor is entirely composed of copper.

12. The system of claim 7, wherein the tantalum includes tantalum pentoxide.

13. The system of claim 7, wherein the implantable medical device is an implantable blood pump.

14. The system of claim 13, wherein the implantable blood pump is electrically coupled to a controller implanted within the body, the controller being configured to control operation of the implantable blood pump.

15. The system of claim 14, wherein the controller is electrically coupled to the receiving coil.

16. The system of claim 15, wherein the controller is powered by the receiving coil.

17. The system of claim 7, wherein the receiving coil is disposed in a non-hermetic package.

18. The system of claim 7, wherein each of the plurality of conductors is coated with graphene.

19. The system of claim 7, wherein receiving coil does not include welds and joints.

20. A transcutaneous energy transfer system for powering an implantable blood pump, comprising:
a substantially planar source coil positionable on a patient's skin;
a battery electrically coupled to the source coil, the source coil being configured to transfer electrical energy through the patient's skin into a body of the patient;
a receiving coil implantable within the patient, the receiving coil being configured to receive the energy transferred by the source coil, the receiving coil including a plurality of copper conductors defining a substantially planar coil and configured to power and electrically couple with the implantable blood pump, each the plurality of copper conductors being coated with tantalum pentoxide and defining a Litz configuration without welds and joints; and
a controller implantable within the patient and electrically coupled to the battery and to the receiving coil, the controller configured to control operation of the implantable blood pump.
